(19) United States
(12) Patent Application Publication
(10) Pub. No.: US 2024/0216453 A1
(43) Pub. Date: Jul. 4, 2024

(54) EXTRACTION OF COMPOUNDS WITH BETA-CARYOPHYLLENE, COMPOSITIONS AND USES THEREOF

(71) Applicant: Synaptix Inc., Leeward Highway, Providenciales (TC)

(72) Inventors: Randall E. MIELKE, Newport, OR (US); Randall E. MIELKE, II, South Beach, OR (US); Jeremy Bruce COWIE, Calgary (CA); Rupert D. HOLMES-SMITH, Okanagan Falls (CA)

(73) Assignee: Synaptix Inc., Leeward highway, Providenciales (TC)

(21) Appl. No.: 18/538,894

(22) Filed: Dec. 13, 2023

Related U.S. Application Data
(60) Provisional application No. 63/433,722, filed on Dec. 19, 2022.

(51) Int. Cl. A61K 36/258; A61K 31/00; A61K 31/05; A61K 31/191; A61K 36/23; A61K 36/324; A61K 36/53; A61K 45/06; A61K 47/44

(52) U.S. Cl. CPC A61K 36/258; A61K 31/05; A61K 31/191; A61K 31/658; A61K 36/23; A61K 36/324; A61K 36/53; A61K 45/06; A61K 47/44; A61K 2236/31; A61K 2236/53

(57) ABSTRACT

Provided are methods for extracting one or more target compounds, such as diterpenes, diterpenoids, triterpenes, and triterpenoids, from a natural biomass using beta-caryophyllene as an extraction solvent. Also provided are compositions comprising beta-caryophyllene, and optionally black cumin seed oil, each independently as a carrier, a delivery adjuvant, a pharmaceutical active ingredient, or any combination thereof; and a diterpene, a diterpenoid, a triterpene, a triterpenoid, or any combination thereof terpenes or triterpenes, including such compositions prepared by the methods herein. Optionally, the compositions further comprise a flavouring agent and/or one or more cannabinoids. Also provided are uses of the pharmaceutical compositions in the treatment of a disease, a disorder or a condition.

### FIELD OF THE DISCLOSURE

The present disclosure relates generally to methods for extraction of target compounds such as diterpenes and triterpenes using beta-caryophyllene, and use of beta-caryophyllene, and optionally black cumin seed oil, each independently as a carrier, delivery adjuvant and/or pharmaceutical active ingredient.

### BACKGROUND

Many extracting solvents can negatively affect people and ecosystems through repeated exposures. Exposure to these extraction solvents can be toxic and can remain present for long periods of time. Hazardous extraction solvents from multiple sources can disseminate over both long and short-terms and into all facets of the environment. Biological organisms, both terrestrial and aquatic, can accumulate toxic levels of these solvents with daily incidental exposures through direct physical contact and/or through food chain consumption. Most importantly, these toxic extracting organic solvents have also been found to be present in humans.

Organic solvents are considered persistent pollutants due to their resistance to environmental degradation through biological, photolytic, or by any other natural chemical process. The bioaccumulation of persistent pollutants in organisms can cause a wide range of environmental health issues. There is a large variety of reported ailments relating to organic chemical exposures including organ malfunction, neurological dysfunction, Alzheimer's disease, autism, disruption of the endocrine system, and even cancer. Such ailments can often lead to mental impairment, severe sickness, or sometimes even death. Most often, it is difficult to identify these health issue-causing contaminants since our mobility inherently results in increased exposure times to many types of chemicals in our everyday activities.

In the US alone, synthetic organic chemicals make up of more than 80,000 distinct chemicals that biological organisms are possibly coming into contact with on a daily basis, while safety testing of only a small fraction of these have been completed. In particular, there are concerns regarding international borders not being respected when chemical pollution is involved. Cost and complexity are the major obstacles for understanding the point sources of synthetic chemical contamination resulting in the direct and indirect cause of health challenges for humans and organisms in the environment.

Synthetic organic solvents are used in many existing chemical processes throughout the world, including in the pharmaceutical industry. While synthetic organic extraction solvents can be removed from the final products by additional cleaning steps, both chemical and physical, these steps ultimately result in increased cost and additionally are time consuming steps that may reduce the amount or quality of the final target products.

It is thus an object of the present disclosure to provide improved methods for the extraction of target compounds, and provide products to reduce daily exposure to possibly hazardous and/or toxic synthetic organic solvents.

### SUMMARY

The present disclosure relates to methods utilizing beta-caryophyllene for the extraction of one or more target compounds, such as for example triterpenes.

In an embodiment, the present disclosure relates to a method for extraction of compounds, said method comprising: providing a beta-caryophyllene extraction solvent; combining the beta-caryophyllene extraction solvent with a sample; and selectively removing and/or separating one or more target compounds from the sample using the beta-caryophyllene extraction solvent to provide an extract composition.

In an embodiment, the sample is a natural biomass, such as for example a biomass comprising leaves, flower, resin, bark, seed, roots, tubers, rhizomes, or any combination thereof.

In an embodiment of the methods herein, the step of selectively removing and/or separating one or more target compounds from the sample comprises dissolving the one or more target compounds in the beta-caryophyllene extraction solvent.

In an embodiment of the methods herein, the beta-caryophyllene extraction solvent is maintained in the extract composition as a carrier, a delivery adjuvant, a pharmaceutical active ingredient, or any combination thereof.

In an embodiment, the target compound is a diterpene, a diterpenoid, a triterpene, a triterpenoid, or any combination thereof. In a particular embodiment, the target compound is the triterpene, such as for example a tetracyclic or a pentacyclic triterpene.

In an embodiment, the sample is from Boswellia serrata and the one or more target compounds may be, for example, one or more of 11-Keto-beta-Boswellic acid, 3-O-Acetyl 11-beta-Boswellic acid, alpha-Boswellic acid, beta-Boswellic acid, 3-O-Acetyl 11-keto-alpha-Boswellic acid, 3-O-Acetyl-beta-Boswellic acid, 11-hydroxy-boswellic acid, 3-hydroxytirucallic acid isomer, 3-oxo-tirucallic acid, and 3-O-acetoxy-tirucallic acid isomer.

In an embodiment, the sample is from Centella asiatica and the one or more target compounds may be, for example, Asiatic Acid.

In an embodiment, the sample is from Holy Basil and the one or more target compounds may be, for example, one or more of Ursolic Acid, Oleonolic Acid, 23-Hydroxy-Ursolic Acid, epi-Oleonolic Acid, Betulin, and Betulinic Acid.

In an embodiment, the sample is from Banaba Leaf and the one or more target compounds may be, for example, Corosolic Acid.

In an embodiment, the sample is from Cannabis Root and the one or more target compounds may be, for example, one or more of Friedelanol and Friedelin.

In an embodiment, the sample is from American Ginseng and the one or more target compounds may be, for example, Lupeol.

In an embodiment, the sample is from Olive pomace and the one or more target compounds may be, for example, one or more of Maslinic Acid, 11-oxy-Maslinic Acid, 2-O-Acetyl-Maslinic Acid.

In an embodiment of the methods herein, the combining step comprises mixing the beta-caryophyllene extraction solvent and the sample in an open or a closed system for a defined period of time.

In an embodiment, the mixing step comprises stirring. In an embodiment, the mixing step uses a Soxhlet system.

In an embodiment, the mixing step is carried out between 0° C. and 245° ° C., more particularly between 0° C. and 100° C., and more particularly still is carried out at 20° C. In an embodiment, the mixing is at ambient pressure or lower than ambient pressure.

In an embodiment, the methods herein further comprise a step of purifying the extract composition by filtration, centrifugation or other separation procedure to separate the one or more target compounds from any insoluble materials.

In an embodiment, the present disclosure relates to a pharmaceutical composition produced by the method as disclosed herein, the pharmaceutical composition comprising the extract composition of the beta-caryophyllene extraction solvent and one or more target compounds.

In an embodiment, the present disclosure relates to a pharmaceutical composition comprising: beta-caryophyllene as a carrier, a delivery adjuvant, a pharmaceutical active ingredient, or any combination thereof; and a diterpene, a diterpenoid, a triterpene, a triterpenoid, or any combination thereof.

In an embodiment, the pharmaceutical compositions disclosed herein comprise the beta-caryophyllene in an amount of 50% to 100% (wt/wt) of the composition, more particularly in an amount of 80% to 100% (wt/wt) of the composition.

In an embodiment, the pharmaceutical compositions disclosed herein comprise diterpene, diterpenoid, triterpene, triterpenoid, or combination thereof in an amount of 0.0001% to 50% (wt/wt) of the composition.

In an embodiment, the pharmaceutical compositions as disclosed herein comprise one or more triterpenes and triterpenoids selected from 11-Keto-beta-Boswellic acid, 3-O-Acetyl 11-keto-beta-Boswellic acid, alpha-Boswellic acid, beta-Boswellic acid, 3-O-Acetyl 11-keto-alpha-Boswellic acid, 3-O-Acetyl-beta-Boswellic acid, 11-hydroxy-boswellic acid, 3-hydroxytirucallic acid isomer, 3-oxo-tirucallic acid, and 3-O-acetoxy-tirucallic acid isomer.

In an embodiment, the pharmaceutical compositions as disclosed herein comprise one or more triterpenes and triterpenoids selected from Asiatic Acid.

In an embodiment, the pharmaceutical compositions as disclosed herein comprise one or more triterpenes and triterpenoids selected from Ursolic Acid, Oleonolic Acid, 23-Hydroxy-Ursolic Acid, and epi-Oleonolic Acid, Betulin and Betulinic Acid.

In an embodiment, the pharmaceutical compositions as disclosed herein comprise one or more triterpenes and triterpenoids selected from Corosolic Acid.

In an embodiment, the pharmaceutical compositions as disclosed herein comprise one or more triterpenes and triterpenoids selected from Friedelanol and Friedelin.

In an embodiment, the pharmaceutical compositions as disclosed herein comprise one or more triterpenes and triterpenoids selected from Lupeol.

In an embodiment, the pharmaceutical compositions as disclosed herein comprise one or more triterpenes and triterpenoids selected from Maslinic Acid, 11-oxy-Maslinic Acid, and 2-O-Acetyl-Maslinic Acid.

A pharmaceutical composition comprising: beta-caryophyllene as a carrier, a delivery adjuvant, a pharmaceutical active ingredient, or any combination thereof; a carrier oil; and one or more of an essential oil, a monoterpene, or any combination thereof, as a flavouring agent. In an embodiment, the carrier oil is black cumin seed oil. In an embodiment, the carrier oil in an amount of between about 0.1% and about 50% (wt/wt), between about 5% and about 30% (wt/wt), or between about 5% and about 15% (wt/wt).

In an embodiment, the pharmaceutical compositions as disclosed herein comprise beta-caryophyllene in an amount of 20% to 99% (wt/wt) of the composition, 60% to 90% (wt/wt) of the composition, or 75% to 85% (wt/wt) of the composition.

In an embodiment, the pharmaceutical compositions disclosed herein that comprise a carrier oil, are substantially free of any triterpene and triterpenoid, or are free of any triterpene and triterpenoid. In an embodiment, the compositions further comprise a diterpene, a diterpenoid, a triterpene, a triterpenoid, or any combination thereof. In an embodiment, the diterpene, diterpenoid, triterpene, triterpenoid, or combination thereof is in an amount of 0.1% to 50% (wt/wt) of the composition.

In an embodiment, the pharmaceutical compositions as disclosed herein comprise one or more triterpenes and triterpenoids selected from boswellic acids. In an embodiment, the pharmaceutical compositions comprise the boswellic acids in an amount from about 0.1% to about 10% (wt/wt).

In an embodiment, the pharmaceutical compositions as disclosed herein comprise one or more triterpenes and triterpenoids selected from 11-Keto-beta-Boswellic acid, 3-O-Acetyl 11-keto-beta-Boswellic acid, alpha-Boswellic acid, beta-Boswellic acid, 3-O-Acetyl 11-keto-alpha-Boswellic acid, 3-O-Acetyl-beta-Boswellic acid, 11-hydroxy-boswellic acid, 3-hydroxytirucallic acid isomer, 3-oxo-tirucallic acid, and 3-O-acetoxy-tirucallic acid isomer.

In an embodiment, the pharmaceutical compositions as disclosed herein comprise one or more triterpenes and triterpenoids selected from Asiatic Acid. In an embodiment, the pharmaceutical compositions as disclosed herein comprise one or more triterpenes and triterpenoids selected from Ursolic Acid, Olconolic Acid, 23-Hydroxy-Ursolic Acid, and epi-Olconolic Acid, Betulin and Betulinic Acid. In an embodiment, the pharmaceutical compositions as disclosed herein comprise one or more triterpenes and triterpenoids selected from Corosolic Acid. In an embodiment, the pharmaceutical compositions as disclosed herein comprise one or more triterpenes and triterpenoids selected from Friedelanol and Friedelin. In an embodiment, the pharmaceutical compositions as disclosed herein comprise one or more triterpenes and triterpenoids selected from Lupeol. In an embodiment, the pharmaceutical compositions as disclosed herein comprise one or more triterpenes and triterpenoids selected from Maslinic Acid, 11-oxy-Maslinic Acid, and 2-O-Acetyl-Maslinic Acid.

In an embodiment, the pharmaceutical compositions as disclosed herein comprise an essential oil blend as a flavouring agent. In an embodiment, the essential oil blend is in an amount from about 0.1% to about 5%. In an embodiment, the essential oil blend comprises lavender oil, lemongrass oil, clove oil, black pepper oil, eucalyptus oil, or any combination thereof. In an embodiment, the essential oil blend comprises lavender oil, lemongrass oil, clove oil, black pepper oil, and eucalyptus oil.

In an embodiment, the present disclosure relates to a pharmaceutical composition comprising between about 70% and about 95% (wt/wt) beta-caryophyllene; between about 5% and about 30% black cumin seed oil; and between about 0.1% and about 2% of one or more essential oils.

In an embodiment, the present disclosure relates to a pharmaceutical composition comprising between about 75% and about 85% (wt/wt) beta-caryophyllene; between about 0.1% and about 10% (wt/wt) boswellic acids; between about 5% and about 15% black cumin seed oil; and between about 0.1% and about 2% of one or more essential oils.

In an embodiment, the pharmaceutical compositions disclosed herein further comprising one or more cannabinoids. In an embodiment, the one or more cannabinoids comprise CBD, CBD-a, THC, THC-a, CBG, CBG-a, CBN, or any combination thereof.

In an embodiment, the present disclosure relates to the use of the pharmaceutical compositions as disclosed herein in the treatment or prevention of a disease, a disorder or a condition.

In an embodiment, the present disclosure relates to the use of the pharmaceutical compositions as disclosed herein in the treatment of diabetes, more particularly Type II diabetes, and/or in modulating blood glucose levels in a subject.

In an embodiment, the present disclosure relates to the use of the pharmaceutical compositions as disclosed herein for remediation of effects caused by intake of THC in a subject. In an embodiment, for the use in remediation of effects caused by intake of THC, the pharmaceutical composition further comprises CBD. In an embodiment, the remediation of effects caused by intake of THC comprises remediation of psychotropic effects of THC. In an embodiment, the remediation of effects caused by intake of THC is due to overconsumption of THC by the subject.

Other aspects and embodiments of the disclosure are evident in view of the detailed description provided herein

### DETAILED DESCRIPTION

The present disclosure provides methods for extraction of target compounds, such as diterpenes, diterpenoids, triterpenes and triterpenoids using beta-caryophyllene. As well, the present disclosure relates to pharmaceutical compositions comprising beta-caryophyllene as a carrier, a delivery adjuvant and/or a pharmaceutical active ingredient in combination with one or more other compounds, such as diterpenes, diterpenoids, triterpenes, or triterpenoids; a carrier oil; and/or one or more of an essential oil, a monoterpene, or any combination thereof.

It is an objective of the present disclosure to provide a 100% natural, plant based product developed as an active ingredient for formulation into finished products, such as in the pharmaceutical compositions disclosed herein. In an embodiment, the products disclosed herein may comprise purely concentrated plant actives and botanical extracts, in which in certain embodiments each ingredient is verified to be safe having undergone significant scientific study and a record of well-established use.

It is an objective of the present disclosure to provide a pharmaceutical composition that is relatively inexpensive and time-effective, such as for direct application to specific health issues. For example, the pharmaceutical composition disclosed herein may be used in a wide range of therapeutic uses covering product categories for e.g. cannabis overdose treatment, managing the effects of THC, pain control, inflammation, immunity, oxidative stress management, addiction treatment, cancer therapy, digestive health, mental health and brain health. In an embodiment, the pharmaceutical composition may be used for health issues that arise by the intentional or unintentional use of illicit drugs.

It is a further objective of the present disclosure to provide chemically pure ingredients that can be used without fear of synthetic organic solvents being present as a result of the extraction process.

It is a further objective of the present disclosure to provide a dependable extraction method that can be replicated throughout the world to meet demand, for example as organic terpenes become a modern day health additive to foods and through transdermal applications.

It is a further objective of the present disclosure to specifically utilize a naturally occurring chemical terpene, i.e. beta-caryophyllene, as the extraction solvent for the unique and selective removal of target natural diterpenes, diterpenoids, triterpenes and other biologically active compounds, such as for example those present in Boswellia powder.

It is a further objective of the present disclosure to provide for the consistent composition of the pharmaceutically active natural ingredients that can be used in pharmacologically active formulations.

It is a further objective of the present disclosure to utilize a beta-caryophyllene extraction solvent as the primary carrier medium for the extracted target compounds. For example, in an embodiment an extract composition comprising beta-caryophyllene and Boswellic acid is disclosed which provides potent nutraceutical ingredients with unique composition and properties, including the beta-caryophyllene. In select embodiments, the present disclosure provides pharmaceutical compositions comprising beta-caryophyllene as a carrier, a delivery adjuvant, a pharmaceutical active ingredient, or any combination thereof. In further select embodiments, the present disclosure provides pharmaceutical compositions comprising beta-caryophyllene and black cumin seed oil, each independently as a carrier, a delivery adjuvant, a pharmaceutical active ingredient, or any combination thereof. In select embodiments, the compositions herein comprise a carrier oil. In select embodiments, the compositions herein comprise one or more of an essential oil, a monoterpene, or any combination thereof.

It is a further objective of the present disclosure to provide a method for the extraction of Boswellic acid from Boswellia powder using a naturally occurring terpene solvent, i.e. beta-caryophyllene extraction solvent. In an embodiment, such methods herein are used to form unique terpene-based pharmacological nutraceutical formulations for treating a wide range of inflammatory and associated health conditions. Without being bound to theory, in the methods disclosed herein the beta-caryophyllene acts as a preferential extractant because it is an aliphatic compound with a unique bicyclic structure and the beta-caryophyllene is unique as extractant solvent because it does not need to be removed and may further act as a pharmacological ingredient in the final product formulation.

In an embodiment, the present disclosure relates to a method for extraction of compounds, said method comprising: providing a beta-caryophyllene extraction solvent; combining the beta-caryophyllene extraction solvent with a sample; and selectively removing and/or separating one or more target compounds from the sample using the beta-caryophyllene extraction solvent to provide an extract composition.

As used herein, “beta-caryophyllene extraction solvent” is used to refer to an extraction solvent comprising beta-caryophyllene. In an embodiment, the beta-caryophyllene extraction solvent comprises at least 75% by weight beta-caryophyllene, more particularly at least 90% by weight beta-caryophyllene, and more particularly still at least 95% by weight beta-caryophyllene. In an embodiment, the beta-caryophyllene extraction solvent consists of beta-caryophyllene (i.e. about 100% beta-caryophyllene).

Beta-caryophyllene (“BCP”) is a known anti-inflammatory and anti-viral agent. BCP has been studied for its medicinal properties and therapeutic benefits in the areas of immunity, pain management, inflammation, oxidative stress, addiction treatment, wound care, cancer therapy, brain health, mental health, and digestive health. BCP has been known since 1834 and was first isolated by Wallach in 1892. Below is the chemical formula of a BCP molecule:

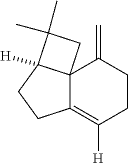

BCP is approved by the United States Food and Drug Administration (FDA) as a “generally recognized as safe” (GRAS) food or cosmetic additive.

As disclosed herein, BCP was found to have unique solvent properties in the selective extraction of compounds from a sample, e.g. a natural biomass. Without being bound by theory, this may be based on the unusual bicyclic structure of BCP, which includes a cyclobutane ring and a C-9 ring with an olefin moiety, as well as a double bond exo to the rings. Advantageously, as reported herein, BCP is well suited to selectively extract lower polarity, higher molecular weight triterpenes, especially tetracyclic and pentacyclic triterpenes.

BCP is a naturally occurring bicyclic sesquiterpene that can be obtained from (e.g. in essential oils) from many different plant sources, including but not limited to Cannabis sativa (cannabis), Cinnamomum spp. (cinnamon), Copaifera officinalis (Copaiba tree), Humulus lupulus (hops), Lavandula angustifolia (lavender), Ocimum spp. (basil), Origanum vulgare L. (oregano), Piper nigrum L. (black pepper), Rosmarinus officinalis (rosemary), and Syzygium aromaticum (cloves). BCP may be steam distilled from stems and the flowering bodies using a conventional steam distillation method. The BCP compound itself is an important pharmacological compound in many health related illnesses.

Copaiba oil that is derived from holes drilled into the Copaifera langsdorffii (Copaiba Tree, also known as Copaifera officinalis) tree trunk allows sap to be gathered in external containers. The sap generated is called balsam which contains moderate concentration of BCP. The balsam may be further concentrated by steam distillation to generate BCP concentrations between 50% and 60%. The Copaiba tree grows in the tropical regions of the world, but the balsam is predominately harvested in South American countries such as Brazil, Venezuela and Guvana.

Advantageously, the present disclosure utilizes BCP's unique chemical extracting properties, as reported herein, for use in extraction of target compounds, including important triterpenes, such as for example Boswellic Acids in Boswellia Powder, from sources throughout the world including plants, biological organisms and other possible traps (e.g. soils and water bodies).

Advantageously, the methods of extraction disclosed herein utilize a BCP extraction solvent (i.e. a nature-derived organic solvent of BCP) that will isolate one or more target compounds to provide an extract composition that will not contain the synthetic possibly hazardous organic solvents that are presently being utilized throughout the world. The extract composition may, in turn, be utilized in the pharmaceutical compositions disclosed herein.

The methods herein comprise a step of combining the beta-caryophyllene extraction solvent with a sample. The beta-caryophyllene extraction solvent may be combined with the sample by any suitable means. In an embodiment, the beta-caryophyllene extraction solvent and sample are combined by mixing. In an embodiment, the mixing may be in an open or a closed system. The combining may be for a defined period of time, such as for between 30 minutes and 48 hours. In an embodiment, the defined period of time is about 30 min, about 1 hour, about 3 hours, about 6 hours, about 12 hours, about 18 hours, about 24 hours, about 36 hours, or about 48 hours.

In an embodiment, the mixing step comprises stirring. In an embodiment, the mixing step uses a Soxhlet system.

The combining may be performed at any suitable temperate. In an embodiment, the combining step is carried out at between 0° ° C. and 245° C., more particularly between 0° C. and 100° C., and more particularly still is carried out at 20° C. In an embodiment, the mixing is at ambient pressure or lower than ambient pressure.

As used herein, the term “sample” is intended to refer to any substance or composition of matter from which it is desirable to perform extraction therefrom. In an embodiment, the sample is a natural biomass, such as a plant material. The natural biomass may be any part of a plant material or a substance or composition derived therefrom. In an embodiment, the natural biomass comprises leaves, flower, resin, bark, seed, roots, tubers, rhizomes, or any combination thereof.

In an embodiment and without limitation, the sample may be from Boswellia Serrata, Centella Asiatica, Holy Basil, Banaba Leaf, Cannabis Root, American Ginseng, Olive pomace, or any combination thereof.

The methods of the present disclosure are capable of removing and/or separating one or more target compounds from the sample. In an embodiment, the target compound is a diterpene, a diterpenoid, a triterpene, a triterpenoid, or any combination thereof. In a particular embodiment, the target compound is the triterpene, such as for example a tetracyclic or a pentacyclic triterpene.

Triterpenes are an extensive group of natural compounds with a broad range of pharmacological properties, such as cytotoxic, anti-cancer, anti-inflammatory, anti-oxidative, anti-bacterial, anti-fungal, anti-viral and wound healing activity.

In an embodiment, the one or more target compounds may be, for example, one or more of 11-Keto-beta-Boswellic acid, 3-O-Acetyl 11-beta-Boswellic acid, alpha-Boswellic acid, beta-Boswellic acid, 3-O-Acetyl 11-keto-alpha-Boswellic acid, 3-O-Acetyl-beta-Boswellic acid, 11-hydroxy-boswellic acid, 3-hydroxytirucallic acid isomer, 3-oxo-tirucallic acid, 3-O-acetoxy-tirucallic acid isomer, Asiatic Acid, Ursolic Acid, Oleonolic Acid, 23-Hydroxy-Ursolic Acid, epi-Oleonolic Acid, Betulin, Betulinic Acid, Corosolic Acid, Friedelanol, Friedelin, Lupeol, Maslinic Acid, 11-oxy-Maslinic Acid, and 2-O-Acetyl-Maslinic Acid.

Boswellic acids (see below chemical formulas) are triterpenes that may be extracted from Boswellia powder or Boswellia resin using the methods disclosed herein employing the beta-caryophyllene extraction solvent.

Boswellia powder, generally called Frankincense oil or olibanum resin, is derived from the Boswellia serrata tree that has been greatly sought after for generations for its medicinal value. The Boswellia resin derived from the pulverizing of the tree bark into a resin, was first mentioned for usage as a drug in the Ebers papyrus, ˜1500 BC. ‘Tears of Horus’ is an early Egyptian myth that described the resin representation, while the Greeks and Romans prized resin was exported to Rome, China and even North Africa. It has been known that the ancient Egyptians used the Boswellia powder resin for mummification balms and unguents. Boswellia resin was used as incense in ancient Judea temple ceremonies and in the Babylonian Talmud (3rd-6th centuries A.D.) it was written that Boswellia powder resin was given to prisoners, condemned to death, to ‘numb the senses’ or ‘not to be sorry’, during their last meal. The drink given to Jesus before his crucifixion is assumed by some scholars to contain the Boswellia powder resin. Traditional medicine has widely accepted use of Boswellia powder resin by Ayurvedic and Chinese cultures. Presently, India uses extracts of Boswellia serrata for treatment of rheumatic diseases and ulcerative colitis, it is being used in mouthwash as antiseptic agents, for the treatment of cough and asthma, and as an added component in detergents, soaps, creams, lotions and perfumes. Frankincense resin is one of the commonly used aromatherapy oils and, additionally, its biological activity is well documented as an anti-inflammatory, antimicrobial and immunomodulatory. In more recent research the BA's presence is showing promising results in the reduction of severity of illnesses resulting from the COVID-19 in the elderly, where the highest death rates are observed.

Below are chemical formulas of Boswellic acids present in boswellia powder, and which may be extracted by the methods disclosed herein:

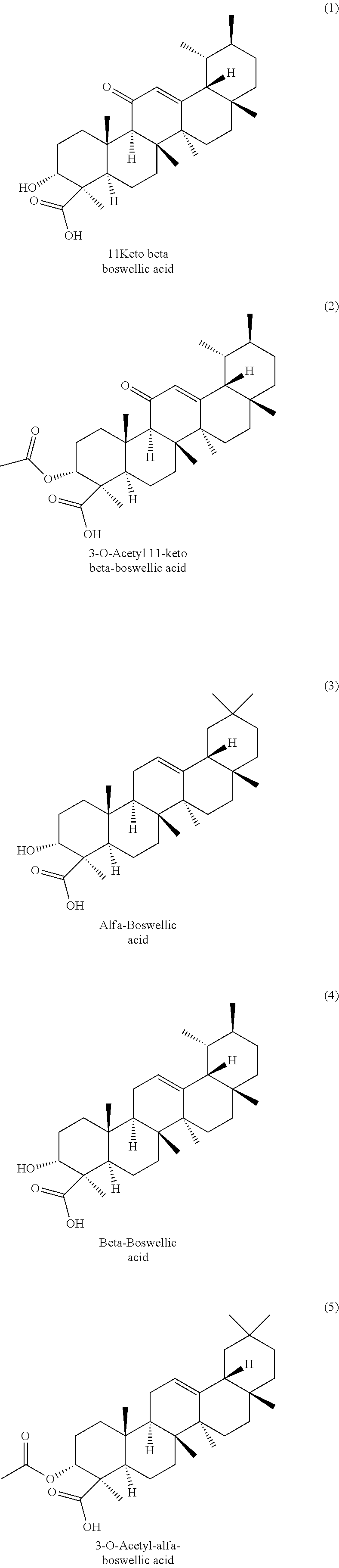

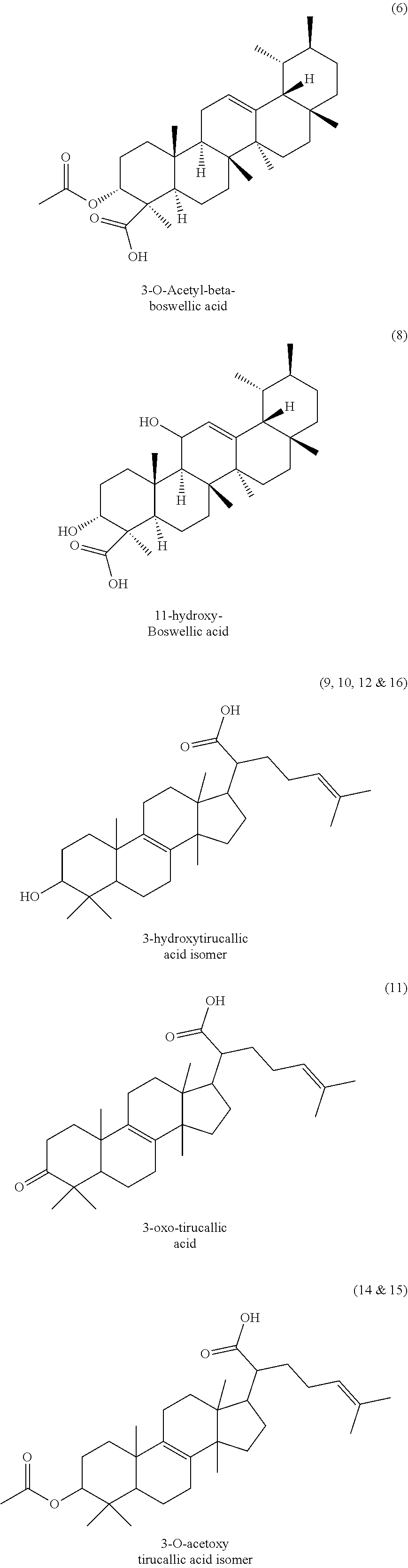

Through the methods disclosed herein, BA is produced through a “green” extraction method that utilizes a plant based solvent as opposed to an industrial synthetic chemical solvent which can leave behind toxic residues. Advantageously, BA produced by the methods herein is purity assured.

BA is known for its anti-inflammatory actions and pain relief properties and has been shown through clinical study to support certain cancer therapies, digestive health, and asthmatic, arthritic, diabetic, Parkinson and Alzheimer's treatments.

Asiatic acid extracted from Centella asiatica (Gotu kola) is a herbaceous perennial plant that grows in wetlands but can be grown in an agricultural setting when watering is regular. The potential pharmacological herb has been used in traditional herbal remedies for centuries, is considered the “Elixir of Life” and has become a focus of scientific research due to its broad range physiological health impacts. Asiatic acid has been shown to be an anti-inflammatory, anti-cancer, wound healing, anti-diabetic, anti-oxidation and hepatoprotective, skin related diseases, cardiovascular diseases, and digestive diseases. Below is the chemical formula of asiatic acid, which may be extracted by the methods disclosed herein;

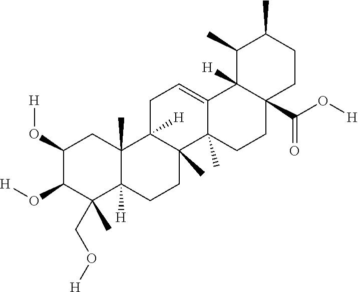

Ursolic acid and other triterpenes have been used in Chinese herbal medicines and are used for many ailments. Ursolic acid was identified in Orthosiphon stamineus, Crataegus monogyna, Lagerstroemia speciosa and Arctostaphylos uva-ursi leaves (7773, 4165, 2108 and 1034 mg/kg, respectively) at concentrations that make them a great renewable source of Ursolic acid. Additional sources of Ursolic acid have been analyzed at various concentrations, including rosemary (Rosmarinus officinalis) leaves, marjoram (Origanum majorana) leaves, oregano (Origanum vulgare) leaves, thyme (Thymus vulgaris) leaves, lavender (Lavandula angustifolia) leaves and flowers, eucalyptus (Eucalyptus) leaves and bark, black elder (Sambucus nigra) leaves and bark, hawthorn (Crataegus spp.) leaves and flowers, coffee (Coffea arabica) leaves, and fruits apple peels (Malus domestica).

Ursolic acid may be utilized as a natural remedy for human and animal consumption. There are promising results for its use in the treatment of various ailments, including cancer, diabetes, cardiovascular diseases, brain diseases, liver diseases, and skeletal diseases. Below is the chemical formula of an ursolic acid molecule, which may be extracted by the methods disclosed herein:

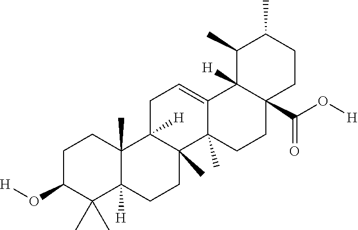

Betulinic acid is a triterpene that is found in many varieties of plants from ground covers to evergreen and birch trees. The variety of sweet basil (Ocimum basilicum L. (Lamiaceae) is rich in Betulinic acid and has shown great potential as a medicinal herbal source for treatment of many human illnesses. Betulinic acid is also present in birch (Betula pendula) trees, buckhorn Ziziphus spp. (Rhamnaceae), Indian blackberry Syzygium spp. (Myrtaceae), persimmon Diospyros spp. (leucomelas) and Peony Paeonia spp. (Paeoniaceae). Betulinic acid has the potential as a herbal remedy to many ailments, including as an anti-inflammatory, anti-microbial, anti-HIV, anti-cancer, and anti-malarial. Below is the chemical formula of a betulinic acid molecule, which may be extracted by the methods disclosed herein:

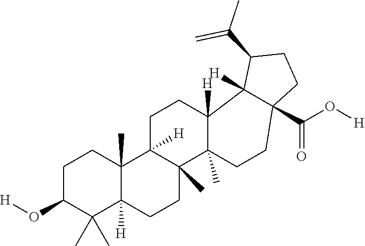

Corosolic acid is predominantly found at elevated concentration in Banaba (Lagerstroemia speciosa L.). Corosolic acid is a natural herbal remedy for diabetes and as research grows it will be determined to impact other diseases. The uses studied so far include anti-diabetic, osteoblast stimulation, colon cancer, urinary infections, and anti-oxidant. Below is the chemical formula of a Corosolic acid molecule, which may be extracted by the methods disclosed herein:

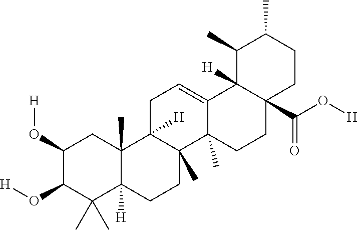

Friedelanol and Friedelin are highly researched triterpenes and have been found in many plants. One source, that is a uniquely large waste product, is the roots from cannabis plants (Cannabis sativa L.). With the popularity and legalization of cannabis, these roots will become a huge reservoir that can be exploited. Below are the chemical formulas of the Friedelinol (left) and Friedelin (right) molecules, which may be extracted by the methods disclosed herein:

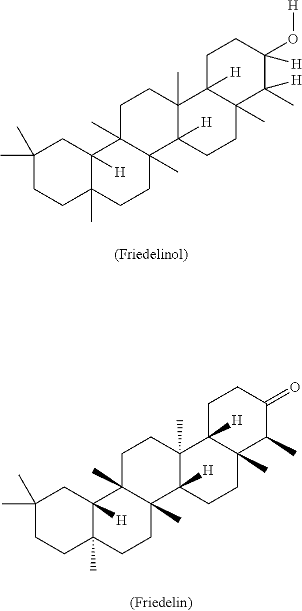

Lupeol is found in many plant species including the American ginseng (Panax quinquefolium L.) and has many pharmacological compounds, namely Ginsenosides. The ability for Lupeol to be an essential medicinal herbal triterpene is becoming clear with the increase in research into a variety of health ailments. These health ailments include the following, without limitation: anti-inflammatory, anti-arthritis, anti-cancer, anti-oxidant, anti-cholesterol, wound healing, anti-angiogenic, anti-microbial, and other ailments. Below is the chemical formula of a Lupeol molecule, which may be extracted by the methods disclosed herein:

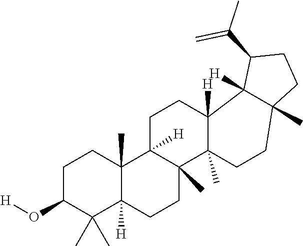

Maslinic acid was isolated from Crataegus oxyacantha L. and was initially named “crategolic acid” in 1927. As research progressed, Maslinic acid (MA) was found in husks of olives and it was determined that dried husks contained higher concentrations and is a great reservoir to isolate a triterpene from a waste product. The source of Maslinic acid in olive (Olea europaea L.) has been shown to have varied concentration levels and is also found in varied legumes and other plant sources. The ailments that Maslinic acid has been researched to impact is starting to grow as the benefits become elucidated. The uses include the following, without limitation: anti-inflammatory, anti-oxidative, anti-cancer, colon cancer, anti-oxidant, anti-neurotoxicity, anti-diabetic, anti-malarial and anti-HIV. Below is the chemical formula of a Maslinic acid molecule, which may be extracted by the methods disclosed herein:

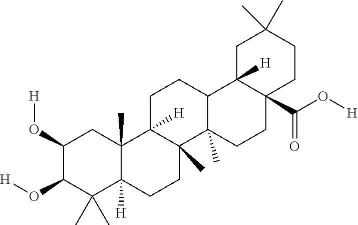

The present disclosure provides a solution and unique technology for extracting pharmacologically specific organic chemicals utilizing a natural compound, BCP. BCP extraction uses a contaminant-free solvent from biomass extraction, which is advantageous for various compositions and uses, including those involving transdermal application methods (described below) in that they are capable of providing the health benefits without the problematic issue of toxic solvents remaining in the final products.

In an embodiment, the methods herein involve the following steps:

1. Providing a beta-caryophyllene extraction solvent. In an embodiment, the beta-caryophyllene extraction solvent comprises at least 75% by weight beta-caryophyllene, more particularly at least 90% by weight beta-caryophyllene, and more particularly still at least 95% by weight beta-caryophyllene. In an embodiment, the beta-caryophyllene extraction solvent comprises about 90%, about 91%, about 92%, about 93%, about 94%, about 95%, about 96%, about 97%, about 98%, or about 99% by weight beta-caryophyllene. In an embodiment, the beta-caryophyllene extraction solvent comprises 98% by weight beta-caryophyllene. In an embodiment, the beta-caryophyllene extraction solvent consists of beta-caryophyllene (i.e. 100% beta-caryophyllene).

2. Combining the beta-caryophyllene extraction solvent with a sample to form a mixture. In an embodiment, the combining is by mixing, e.g. in an open or closed system. In an embodiment, the mixing is stirring. In an embodiment, in the mixture comprises between about 5% and about 50% by weight of the sample, more particularly between about 10% and about 35% by weight of the sample. In an embodiment, the mixture comprises between about 50% and about 95% by weight of the beta-caryophyllene extraction solvent, more particularly between about 65% and about 90% by weight of the beta-caryophyllene extraction solvent. In an embodiment, the step of combining the beta-caryophyllene extraction solvent with the sample includes heating of the mixture. In an embodiment, the mixture is heated to a temperature of between about 50° ° C. and about 90° ° C.

3. Selectively removing and/or separating one or more target compounds from the sample using the beta-caryophyllene extraction solvent to provide an extract composition. In an embodiment, the target compounds are removed and/or separated from the sample by dissolving in the beta-caryophyllene extraction solvent and filtering the beta-caryophyllene extraction solvent to separate it from the remainder of the sample. In an embodiment, prior to filtering, the method involves cooling the mixture to room temperature to provide a more viscous solution. In an embodiment, rather than filtering the mixture is left to stand for a period of time, after which the beta-caryophyllene extraction solvent is decanted off the top. The extract composition may then be analyzed (e.g. by Gas Chromatography-Mass Spectrometry (GC-MS)) to determine the concentration and/or amount of target compound extracted from the sample.

For example, in an embodiment the method of extraction disclosed herein comprises: (1) Using 98% beta-caryophyllene extraction solvent, (2) adding approximately 17% by mass of Boswellia powder (65% Boswellic acid) to a stirring jacketed reactor system that has temperature control (+/−1° C. accuracy), (3) heating the reactor to between 70° ° C. to 80° C. which will produce a turbid solution that becomes more viscous on cooling, (4) filtering the solution qualitatively before cooling to reduce extended periods of filtration times upon solution cooling to room temperatures, and (5) analyzing the extract by GC-MS to determine Boswellic acid concentrations. In the methods herein, there may also be an additional step for removing any problematic matrix.

For example, with respect to the extraction of Boswellia powder described herein, an initial qualitative filtration step may be used to remove large particulate matter, including biomass components that remain present from either or both of the beta-caryophyllene extraction solvent and the Boswellia powder. The clarification step may be advantageous when various beta-caryophyllene extraction solvent and Boswellia powder raw materials are not identical in composition from batch to batch (e.g. different shipments and sources). Vacuum assisted Columnar filtration systems using different filtration media may be used to clarify the Boswellic acid extract to meet various product requirement (e.g. clarity requirements). The use of carbon, silica and diatomaceous earth products may be adjusted in volume as determined by the specific problematic matrix issues as the clarification step progresses. GC-MS analysis of clarified Boswellic acids extract may be done after final filtration steps to ensure quantitative Boswellic acid and beta-caryophyllene calculations are available. While exemplary reference is made to Boswellic acid, the procedure can be used for any of the target compound described herein (e.g. diterpenes, diterpenoids, triterpenes and triterpenoids).

The extraction of target compounds from samples using the methods of extraction of the present disclosure is beneficial in providing the bioactive ingredients. Advantageously, the beta-caryophyllene extraction solvent in itself has been shown to enhance biochemical processes that produces increased health benefits.

### Compositions

In an embodiment, the present disclosure provides a pharmaceutical composition produced by the methods as described herein, the pharmaceutical composition comprising the extract composition of the beta-caryophyllene extraction solvent and one or more target compounds.

In an embodiment, the present disclosure provides a pharmaceutical composition comprising: beta-caryophyllene as a carrier, a delivery adjuvant, a pharmaceutical active ingredient, or any combination thereof; and a diterpene, a diterpenoid, a triterpene, a triterpenoid, or any combination thereof.

In an embodiment, the present disclosure provides a pharmaceutical composition comprising: beta-caryophyllene as a carrier, a delivery adjuvant, a pharmaceutical active ingredient, or any combination thereof; a carrier oil; and one or more of an essential oil, a monoterpene, or any combination thereof, as a flavouring agent.

In an embodiment, the pharmaceutical compositions comprise a carrier oil. In an embodiment, as used herein the term “carrier oil” refers to a base oil or vegetable oil that can be used to dilute essential oils and active ingredients before they are used, e.g. applied to the skin or ingested. In an embodiment, the carrier oil is a vegetable oil. In an embodiment, the carrier oil is a fruit oil, such as derived from seeds, kernels or nuts.

In an embodiment, the pharmaceutical compositions comprise the carrier oil in an amount of between about 0.01% and about 50% (wt/wt) of the composition, more particularly between about 0.01% and about 50% (wt/wt) of the composition. In an embodiment, the pharmaceutical compositions disclosed herein comprise the carrier oil in an amount of between about 5% and about 30% (wt/wt). In an embodiment, the pharmaceutical compositions disclosed herein comprise the carrier oil in an amount of between about 5% and about 15% (wt/wt).

In an embodiment, the carrier oil is black seed oil, such as black cumin seed oil. Black seed oil is an extract derived from the seeds of Nigella sativa plant, and commonly known as black cumin. In select embodiments, black seed oil may be extracted using a cold press method ensuring the integrity of the phyto compounds is maintained through to the finished product. Preferably, only the highest quality black seed oil is selected for formulation into the pharmaceutical compositions of the present disclosure, e.g. after undergoing a vetting process and quality control audit.

Black seed oil has established medical use going back thousands of years and in modern times for immune response, fever, cough, bronchitis, influenza, asthma, hypertension, brain health, dizziness, headache, diabetes, inflammation, wound care, eczema, tissue growth, and cancer therapy. Without being bound by theory, the active component of black seed oil is believed to be thymoquinone, which has been studied for its antioxidant, antidiabetic, anticancer, analgesic, anti-inflammatory, antimicrobial, antiviral, antifungal, antibacterial, bronchodilator, anticoagulant, reno, gastro and hepatoprotective effects.

In an embodiment, the pharmaceutical compositions comprise one or more of an essential oil, a monoterpene, or any combination thereof, as a flavouring agent. The essential oil or monoterpene may be any suitable essential oil for the desired purpose and/or for use in combination with other components of the composition. In an embodiment, the pharmaceutical composition comprises a blend of essential oils or a blend of monoterpenes. In an embodiment, the pharmaceutical composition comprises the blend in an amount from about 0.1% to about 5% (wt/wt) of the composition, and more particularly from about 0.1% to about 2% (wt/wt) of the composition.

In an embodiment, the pharmaceutical compositions comprise an essential oil blend of lavender oil, lemongrass oil, clove oil, black pepper oil, eucalyptus oil, or any combination thereof. In an embodiment, the essential oil blend consists of comprises lavender oil, lemongrass oil, clove oil, black pepper oil, and eucalyptus oil. In an embodiment, each of the essential oil is 100% pure.

Within the pharmaceutical compositions of the present disclosure, the resulting terpene entourage extract may be a potent nutraceutical ingredient with unique composition and properties, including enhanced bioavailability of the triterpenes.

In an embodiment, the pharmaceutical compositions of the present disclosure comprise the beta-caryophyllene in an amount of 20% to 100% (wt/wt) of the composition, more particularly 50% to 100% (wt/wt) of the composition, more particularly 80% to 100% (wt/wt) of the composition. In an embodiment, the pharmaceutical compositions herein comprise the beta-caryophyllene in an amount of 20% to 99% (wt/wt) of the composition, and more particularly 60% to 90% (wt/wt) of the composition. In an embodiment, the pharmaceutical compositions herein comprise the beta-caryophyllene in an amount of 70% to 95% (wt/wt) of the composition. In an embodiment, the pharmaceutical compositions herein comprise the beta-caryophyllene in an amount of 75% to 85% (wt/wt) of the composition.

In select embodiments of the present disclosure, the pharmaceutical compositions disclosed herein are substantially free of any triterpene and triterpenoid. By “substantially free”, it is intended to mean that the compositions comprise less than 0.1% (wt/wt) of the composition of total content of triterpenes and triterpenoids. In an embodiment, by substantially free it is meant that the triterpenes and triterpenoisd may be present in trace amounts, whether detectable or not detectable.

In select embodiments of the present disclosure, the pharmaceutical compositions disclosed herein are free of any triterpene and triterpenoid. By “free of any triterpene and triterpenoid”, it is meant that there is no triterpenes or triterpenoids in the composition.

In an embodiment, the pharmaceutical compositions of the present disclosure comprise a diterpene, a diterpenoid, a triterpene, a triterpenoid, or any combination thereof. In an embodiment, the pharmaceutical compositions comprise diterpene, diterpenoid, triterpene, triterpenoid, or a combination thereof in an amount of 0.1% to 50% (wt/wt) of the composition. In an embodiment, the pharmaceutical compositions comprise diterpene, diterpenoid, triterpene, triterpenoid, or a combination thereof in an amount from about 0.1% to about 10% (wt/wt) of the composition.

In an embodiment, the pharmaceutical compositions of the present disclosure comprise one or more triterpenes and triterpenoids selected from boswellic acids. In an embodiment, the pharmaceutical composition comprises the boswellic acids in an amount from about 0.1% to about 10% (wt/wt) of the composition.

In an embodiment, the pharmaceutical compositions of the present disclosure comprise one or more triterpenes and triterpenoids selected from 11-Keto-beta-Boswellic acid, 3-O-Acetyl 11-keto-beta-Boswellic acid, alpha-Boswellic acid, beta-Boswellic acid, 3-O-Acetyl 11-keto-alpha-Boswellic acid, 3-O-Acetyl-beta-Boswellic acid, 11-hydroxy-boswellic acid, 3-hydroxytirucallic acid isomer, 3-oxo-tirucallic acid, and 3-O-acetoxy-tirucallic acid isomer.

In an embodiment, the pharmaceutical compositions of the present disclosure comprise one or more triterpenes and triterpenoids selected from Asiatic Acid.

In an embodiment, the pharmaceutical compositions of the present disclosure comprise one or more triterpenes and triterpenoids selected from Ursolic Acid, Oleonolic Acid, 23-Hydroxy-Ursolic Acid, and epi-Oleonolic Acid, Betulin and Betulinic Acid.

In an embodiment, the pharmaceutical compositions of the present disclosure comprise one or more triterpenes and triterpenoids selected from Corosolic Acid.

In an embodiment, the pharmaceutical compositions of the present disclosure comprise one or more triterpenes and triterpenoids selected from Friedelanol and Friedelin.

In an embodiment, the pharmaceutical compositions of the present disclosure comprise one or more triterpenes and triterpenoids selected from Lupeol.

In an embodiment, the pharmaceutical compositions of the present disclosure comprise one or more triterpenes and triterpenoids selected from Maslinic Acid, 11-oxy-Maslinic Acid, and 2-O-Acetyl-Maslinic Acid.

In a particular embodiment, the pharmaceutical composition of the present disclosure comprises between about 70% and about 95% (wt/wt) beta-caryophyllene; between about 5% and about 30% black cumin seed oil; and between about 0.1% and about 2% of one or more essential oils.

In a particular embodiment, the pharmaceutical composition of the present disclosure comprises between about 75% and about 85% (wt/wt) beta-caryophyllene; between about 0.1% and about 10% (wt/wt) boswellic acids; between about 5% and about 15% black cumin seed oil; and between about 0.1% and about 2% of one or more essential oils.

In an embodiment, the pharmaceutical compositions herein may be for administration by any suitable route, including intravenous, intramuscular, subcutaneous, transdermal, oral, buccal, sublingual, topical, rectal, vaginal, nasal, or ocular. In an embodiment, the pharmaceutical is for administration by injection, transdermal patch, inhalation, pill, tablet, liquid, or other dosage form.

Advantageously, various transdermal application options may allow for the delivery of the beta-caryophyllene and/or target compound (e.g. triterpene) to locations that are specific to the health ailment being targeted.

In an embodiment, the steps for a transdermal application method are as follows: (1) Nasal Mucous Membrane Spray in a small bottle with an aerosolization tip to ensure maximum dispersion in the nasal cavity per the bottles volumetric prescribed pumping action. (2) Rollerball designed using a small bottle with a large surface-area rolling ball tip that allows for a prescribed dosage per linear coverage. (3) Transdermal patch with prescribed dosage that can be determined by the square footage of skin to patch coverage.

In the context of the present disclosure, the concentration of BCP in the extract composition may be standardized using Oil of Copaiba that contains BCP. The Oil of Copaiba may be analyzed by GC-MS along with the extract composition to determine BCP concentration, and dilution parameters may be determined to ensure final product concentrations meet the desired amounts of the pharmaceutical composition. Additional monoterpenes and blends thereof may also be added to flavor the final product.

### Uses

The pharmaceutical compositions of the present disclosure were designed to offer an efficacious and safe, natural based product for treating various conditions, such as for example cannabis overdoses and managing the effects of THC. In embodiments described herein, each ingredient formulated in the pharmaceutical compositions has undergone scientific investigation and/or clinical study evaluation during the formulation and product development phase to verify the safety and pharmacological activity of the pharmaceutical compositions and a multitude of usage applications were identified. The findings elucidate uses for pharmaceutical compositions of the present disclosure in reducing the effects of thc, pain control, immune response, inflammatory conditions, oxidative stress management, cancer therapy, addiction treatment, digestive health, mental health, heart health and brain health.

The components of the pharmaceutical compositions herein have demonstrated analgesic, anti-inflammatory, antipyretic, anticonvulsant, antioxidant, antibacterial, antiviral, antimicrobial, antitumor, anxiolytic, antidepressant, antinociceptive, myorelaxant, antispasmodic pharmacological actions in clinical study and trial. Without being bound by theory, with respect to reducing the psychotropic effects of THC, it is believed that the pharmaceutical compositions of the present disclosure interact with the CB1 receptor to release the THC from the orthosteric binding site.

In an embodiment, the present disclosure relates to the use of the pharmaceutical composition as described herein in the treatment or prevention of a disease, a disorder or a condition.

BCP's involvement with the brain's endocannabinoid receptor (CB2) provides evidence of possible rewarding reactions to many illicit drugs, including Cannabis and Methamphetamine. The research shows reduced craving for illicit drugs, like methamphetamine, and a rapid reduction in the hallucinogenic effects from cannabis (e.g. from tetrahydrocannabinol; THC). As such, pharmaceutical compositions of the present disclosure may be used to reduce the effects of illicit drugs, such as THC, when consumed purposefully in excess or accidentally. Utilizing a broad range of delivery methods, application with BCP and other specified triterpenes may ultimately save lives while potentially also decreasing possible recovery times.

Thus, in an embodiment, the present disclosure relates to the use of the pharmaceutical composition as described herein for remediation of effects caused by intake of THC in a subject. In an embodiment, for the use in remediation of effects caused by intake of THC, the pharmaceutical composition further comprises CBD. In an embodiment, the remediation of effects caused by intake of THC comprises remediation of psychotropic effects of THC. In an embodiment, the remediation of effects caused by intake of THC is due to overconsumption of THC by the subject.

The ability to target a variety of health related illnesses can be assessed by several application methods, including those that are all transdermal in origin. Transdermal application methods can use nasal mucous membranes and the outer skin surfaces to allow the target pharmaceutical compounds (e.g. triterpenes) to move through the capillary beds and onto the specific cell surface receptors being targeted by the compositions of the present disclosure (e.g. containing BCP and Boswellic acid).

In an embodiment, the present disclosure relates to the use of the pharmaceutical composition as described herein in the treatment of diabetes and/or in modulating blood glucose levels in a subject.

### EXAMPLES

### Example 1

Extraction of Boswellic Acids from Boswellia Serrata (Indian Frankincense)

In a 2 L reactor vessel, 130 g of Boswellia Powder (65%) and 1175 g of beta-caryophyllene (>98%) were combined. The mixture was heated to 70° C. while stirring for 2 hours. The mixture was then cooled to room temperature and filtered to provide 1000 g of a clear yellow extract. In an alternate procedure, the mixture was left to stand for 72 hours and then the clear yellow extract was decanted off the top. Analysis of the extract composition showed the presence of triterpenes 5-10%, consisting primarily of Boswellic acids, and beta-caryophyllene 90-95%.

### Example 2

Extraction of Corosolic Acid from Banaba Leaf (Lagerstroemia speciosa L.)

In a 500 ml reactor vessel, 80 g of Banaba Leaf Powder (20%) and 145 g of beta-caryophyllene (>98%) were combined. The mixture was heated to 50° C. while stirring for 2 hours. The mixture was then cooled to room temperature and filtered to provide 110 g of a dark green/brown extract. Analysis of the extract composition showed the presence of triterpenes 5-10%, consisting primarily of Corosolic Acid and Oleanolic Acid, and beta-caryophyllene 90-95%.

### Example 3

Extraction of Ursolic Acid from Holy Basil (Ocimum tenuiflorum)

In a 250 ml reactor vessel, 54 g of Holy Basil and 130 g of beta caryophyllene (>98%) were combined. The mixture was heated to 50ºC while stirring for 2 hours. The mixture was then cooled to room temperature and filtered to provide 75 g of a yellow/orange extract. Analysis of the extract composition showed the presence of triterpenes 1-2%, consisting primarily of Ursolic Acid, and beta caryophyllene 97-98%.

In the present disclosure, all terms referred to in singular form are meant to encompass plural forms of the same. Likewise, all terms referred to in plural form are meant to encompass singular forms of the same. Unless defined otherwise, all technical and scientific terms used herein have the same meaning as commonly understood by one of ordinary skill in the art to which this disclosure pertains.

For the sake of brevity, only certain ranges are explicitly disclosed herein. However, ranges from any lower limit may be combined with any upper limit to recite a range not explicitly recited, as well as, ranges from any lower limit may be combined with any other lower limit to recite a range not explicitly recited, in the same way, ranges from any upper limit may be combined with any other upper limit to recite a range not explicitly recited. Additionally, whenever a numerical range with a lower limit and an upper limit is disclosed, any number and any included range falling within the range are specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values even if not explicitly recited. Thus, every point or individual value may serve as its own lower or upper limit combined with any other point or individual value or any other lower or upper limit, to recite a range not explicitly recited.

Many obvious variations of the embodiments set out herein will suggest themselves to those skilled in the art in light of the present disclosure. Such obvious variations are within the scope of the appended claims.

What is claimed is:

1. A method for extraction of compounds, said method comprising:
providing a beta-caryophyllene extraction solvent;
combining the beta-caryophyllene extraction solvent with a sample; and
selectively removing and/or separating one or more target compounds from the sample using the beta-caryophyllene extraction solvent to provide an extract composition.

2. The method of claim 1, wherein the sample is a natural biomass.

3. The method of claim 2, wherein the natural biomass comprises leaves, flower, resin, bark, seed, roots, tubers, rhizomes, or any combination thereof.

4. The method of claim 1, wherein the step of selectively removing and/or separating comprises dissolving the one or more target compounds in the beta-caryophyllene extraction solvent.

5. The method of claim 4, wherein the beta-caryophyllene extraction solvent is maintained in the extract composition as a carrier, a delivery adjuvant, a pharmaceutical active ingredient, or any combination thereof.

6. The method of claim 5, wherein the target compound is a diterpene, a diterpenoid, a triterpene, a triterpenoid, or any combination thereof.

7. The method of claim 6, wherein the target compound is the triterpene.

8. The method of claim 7, wherein the triterpene is a tetracyclic or a pentacyclic triterpene.

9. The method of claim 1, wherein the sample is from Boswellia Serrata.

10. The method of claim 9, wherein the one or more target compounds is one or more of 11-Keto-beta-Boswellic acid, 3-O-Acetyl 11-keto-beta-Boswellic acid, alpha-Boswellic acid, beta-Boswellic acid, 3-O-Acetyl 11-keto-alpha-Boswellic acid, 3-O-Acetyl-beta-Boswellic acid, 11-hydroxy-boswellic acid, 3-hydroxytirucallic acid isomer, 3-oxo-tirucallic acid, and 3-O-acetoxy-tirucallic acid isomer.

11. The method of claim 1, wherein the sample is from Centella Asiatica.

12. The method of claim 11, wherein the one or more target compounds is Asiatic Acid.

13. The method of claim 1, wherein the sample is from Holy Basil.

14. The method of claim 13, wherein the one or more target compounds is one or more of Ursolic Acid, Oleonolic Acid, 23-Hydroxy-Ursolic Acid, epi-Oleonolic Acid, Betulin, and Betulinic Acid.

15. The method of claim 1, wherein the sample is from Banaba Leaf.

16. The method of claim 15, wherein the one or more target compounds is Corosolic Acid.

17. The method of claim 1, wherein the sample is from Cannabis Root.

18. The method of claim 17, wherein the one or more target compounds is one or more of Friedelanol and Friedelin.

19. The method of claim 1, wherein the sample is from American Ginseng.

20. The method of claim 19, wherein the one or more target compounds is Lupeol.

21. The method of claim 1, wherein the sample is from Olive pomace.

22. The method of claim 21, wherein the one or more target compounds is one or more of Maslinic Acid, 11-oxy-Maslinic Acid, 2-O-Acetyl-Maslinic Acid.

23. The method of claim 1, wherein the combining step comprises mixing the beta-caryophyllene extraction solvent and the sample in an open or a closed system for a defined period of time.

24. The method of claim 23, wherein the mixing step comprises stirring.

25. The method of claim 23, wherein the mixing step uses a Soxhlet system.

26. The method of claim 23, wherein the mixing step is carried out between 0° C. and 100° C.

27. The method of claim 23, wherein the mixing step is carried out at 20° C.

28. The method of claim 23, wherein the mixing step is carried out between 0° ° C. and 245° C., and at ambient pressure or lower than ambient pressure.

29. The method of claim 1, further comprising a step of purifying the extract composition by filtration, centrifugation or other separation procedure to separate the one or more target compounds from any insoluble materials.

30. A pharmaceutical composition produced by the method of 1, the pharmaceutical composition comprising the extract composition of the beta-caryophyllene extraction solvent and one or more target compounds.

31. A pharmaceutical composition comprising:
beta-caryophyllene as a carrier, a delivery adjuvant, a pharmaceutical active ingredient, or any combination thereof; and
a diterpene, a diterpenoid, a triterpene, a triterpenoid, or any combination thereof.

32. The pharmaceutical composition of claim 31, which comprises the beta-caryophyllene in an amount of 50% to 100% (wt/wt) of the composition.

33. The pharmaceutical composition of claim 32, which comprises the beta-caryophyllene in an amount of 80% to 100% (wt/wt) of the composition.

34. The pharmaceutical composition of claim 33, which comprises diterpene, diterpenoid, triterpene, triterpenoid, or combination thereof in an amount of 0.0001% to 50% (wt/wt) of the composition.

35. The pharmaceutical composition of claim 31, which comprises one or more triterpenes and triterpenoids selected from 11-Keto-beta-Boswellic acid, 3-O-Acetyl 11-keto-beta-Boswellic acid, alpha-Boswellic acid, beta-Boswellic acid, 3-O-Acetyl 11-keto-alpha-Boswellic acid, 3-O-Acetyl-beta-Boswellic acid, 11-hydroxy-boswellic acid, 3-hydroxytirucallic acid isomer, 3-oxo-tirucallic acid, and 3-O-acetoxy-tirucallic acid isomer.

36. The pharmaceutical composition of claim 31, which comprises one or more triterpenes and triterpenoids selected from Asiatic Acid.

37. The pharmaceutical composition of 31, which comprises one or more triterpenes and triterpenoids selected from Ursolic Acid, Oleonolic Acid, 23-Hydroxy-Ursolic Acid, and epi-Oleonolic Acid, Betulin and Betulinic Acid.

38. The pharmaceutical composition of claim 31, which comprises one or more triterpenes and triterpenoids selected from Corosolic Acid.

39. The pharmaceutical composition of claim 31, which comprises one or more triterpenes and triterpenoids selected from Friedelanol and Friedelin.

40. The pharmaceutical composition of claim 31, which comprises one or more triterpenes and triterpenoids selected from Lupeol.

41. The pharmaceutical composition of claim 31, which comprises one or more triterpenes and triterpenoids selected from Maslinic Acid, 11-oxy-Maslinic Acid, and 2-O-Acetyl-Maslinic Acid.

42. A pharmaceutical composition comprising:
beta-caryophyllene as a carrier, a delivery adjuvant, a pharmaceutical active ingredient, or any combination thereof;
a carrier oil; and
one or more of an essential oil, a monoterpene, or any combination thereof, as a flavouring agent.

43. The pharmaceutical composition of claim 42, wherein the carrier oil is black cumin seed oil.

44. The pharmaceutical composition of claim 42, which comprises the carrier oil in an amount of between about 0.1% and about 50% (wt/wt).

45. The pharmaceutical composition of claim 42, which comprises the carrier oil in an amount of between about 5% and about 30% (wt/wt).

46. The pharmaceutical composition of claim 42, which comprises the carrier oil in an amount of between about 5% and about 15% (wt/wt).

47. The pharmaceutical composition of claim 42, which comprises the beta-caryophyllene in an amount of 20% to 99% (wt/wt) of the composition.

48. The pharmaceutical composition of claim 42, which comprises the beta-caryophyllene in an amount of 60% to 90% (wt/wt) of the composition.

49. The pharmaceutical composition of claim 42, which comprises the beta-caryophyllene in an amount of 75% to 85% (wt/wt) of the composition.

50. The pharmaceutical composition of claim 42, which further comprises a diterpene, a diterpenoid, a triterpene, a triterpenoid, or any combination thereof.

51. The pharmaceutical composition of claim 50, which comprises the diterpene, diterpenoid, triterpene, triterpenoid, or combination thereof in an amount of 0.1% to 50% (wt/wt) of the composition.

52. The pharmaceutical composition of claim 42, which is substantially free of any triterpene and triterpenoid.

53. The pharmaceutical composition of claim 51, which is free of any triterpene and triterpenoid.

54. The pharmaceutical composition of claim 42, which comprises one or more triterpenes and triterpenoids selected from boswellic acids.

55. The pharmaceutical composition of claim 54, which comprises the boswellic acids in an amount from about 0.1% to about 10% (wt/wt).

56. The pharmaceutical composition of claim 42, which comprises one or more triterpenes and triterpenoids selected from 11-Keto-beta-Boswellic acid, 3-O-Acetyl 11-keto-beta-Boswellic acid, alpha-Boswellic acid, beta-Boswellic acid, 3-O-Acetyl 11-keto-alpha-Boswellic acid, 3-O-Acetyl-beta-Boswellic acid, 11-hydroxy-boswellic acid, 3-hydroxytirucallic acid isomer, 3-oxo-tirucallic acid, and 3-O-acetoxy-tirucallic acid isomer.

57. The pharmaceutical composition of claim 42, which comprises one or more triterpenes and triterpenoids selected from Asiatic Acid.

58. The pharmaceutical composition of claim 42, which comprises one or more triterpenes and triterpenoids selected from Ursolic Acid, Oleonolic Acid, 23-Hydroxy-Ursolic Acid, and epi-Oleonolic Acid, Betulin and Betulinic Acid.

59. The pharmaceutical composition claim 42, which comprises one or more triterpenes and triterpenoids selected from Corosolic Acid.

60. The pharmaceutical composition of claim 42, which comprises one or more triterpenes and triterpenoids selected from Friedelanol and Friedelin.

61. The pharmaceutical composition of claim 42, which comprises one or more triterpenes and triterpenoids selected from Lupeol.

62. The pharmaceutical composition of claim 42, which comprises one or more triterpenes and triterpenoids selected from Maslinic Acid, 11-oxy-Maslinic Acid, and 2-O-Acetyl-Maslinic Acid.

63. The pharmaceutical composition of claim 42, which comprises an essential oil blend as the flavouring agent.

64. The pharmaceutical composition of claim 63, which comprises the essential oil blend in an amount from about 0.1% to about 5%.

65. The pharmaceutical composition of claim 63, wherein the essential oil blend comprises lavender oil, lemongrass oil, clove oil, black pepper oil, eucalyptus oil, or any combination thereof.

66. The pharmaceutical composition of claim 63, wherein the essential oil blend comprises lavender oil, lemongrass oil, clove oil, black pepper oil, and eucalyptus oil.

67. The pharmaceutical composition of claim 42, which comprises:
between about 70% and about 95% (wt/wt) beta-caryophyllene;
between about 5% and about 30% black cumin seed oil; and
between about 0.1% and about 2% of one or more essential oils.

68. The pharmaceutical composition of claim 67, which comprises:
between about 75% and about 85% (wt/wt) beta-caryophyllene;
between about 0.1% and about 10% (wt/wt) boswellic acids;
between about 5% and about 15% black cumin seed oil; and
between about 0.1% and about 2% of one or more essential oils.

69. The pharmaceutical composition of claim 42, further comprising one or more cannabinoids.

70. The pharmaceutical composition of claim 69, wherein the one or more cannabinoids comprise CBD, CBD-a, THC, THC-a, CBG, CBG-a, CBN, or any combination thereof.
